(19) United States
(12) Patent Application Publication
(10) Pub. No.: US 2001/0053349 A1
(43) Pub. Date: Dec. 20, 2001

(54) Artificial nail sanitizing solution

(76) Inventor: Sheila Ann Wright, West Bloomfield, MI (US)

Correspondence Address: Sheila A. Wright DPM, 6370 Village Park Dr - 106, West Bloomfield, MI, 48322, US

(21) Appl. No.: 09/775,577

(22) Filed: Feb. 5, 2001

Related U.S. Application Data
(60) Provisional application No. 60/180,334, filed on Feb. 4, 2000.

(51) Int. Cl. A61K 7/04

(52) U.S. Cl. 424/61

(57) ABSTRACT

A daily perventive method reduces infection risk associated with artificial nails. When applied as a spray or gel soak to the nail and periungal areas, bacterial and fungal counts are significantly reduced. It is a well established medical fact that hand to hand contact is the #1 mode of transmission of infection. Artificial nails act as carriers when applied onto natural fingernails. Attached to the human hand they transmit bacteria one to another on personal contact. The porous composition of artificial nails along with additional gaps and separation created by natural nail growth; provides a template for which skin organisms are trapped and grow. Artificial nails attract water moisture on contact (bandwashing); remaining damp they act as a template which supports growth of organisms.

The utility for this product is referenced by national reports of infant morbity linked to nurses wearing artificial nails, Oklahoma (1999). This pH driven nail sanitizing solution combined with a drying agent significantly reduces bacterial and fungal accummulation. As a proactive approach this antiseptic nail sanitizing solution directly challenges growth requirements of many skin microrganisms.

### BACKGROUND OF THE INVENTION

Normal flora are considered parasites when they invade and multiply in healthy normal tissue as in the nail bed or nail matrix. The role of the intact nailplate as a barrier to infection is paramount. Many topical nail solutions are directed at breeching the nailplate to deliver drugs to treat conditions of early onychia or onychomycosis. The problem here is two fold. Topical nail lacquers in dropper or polish forms are unable to reach deep layers of the nail matrix where fungi and bacteria migrate. Secondly, treatment is usually sought too late to be effective. By the time there is visible evidence of growth the matrix is loaded with organisms. Onychomycosis, onychochauxis nail stripping, trauma are all sequela associated with prolonged use of artificial nails.

Oral compounds containing terbinafine or itraconazole are considered the standard for treatment of documented nail fungus. Clinical and mycological cure rates average 70%. See prior art. Double Blind randomized study of continuous terbinafine compared with intermittent itraconazole in the treatment of toenail onychomycosis. The cost for oral treatment ranges between $700-$800, and the time frame for results averages 9-12 months. Trapped moisture in artifical nails sets up similar conditions as perspiring feet which facilitates the development of fungal toenails. Recently, artificial nails are also placed on toenails.

Today, the focus is on prevention. Artificial nail wearers want clean safe nails. Old treatment modalities ranging from drops, polish, removers, lacquers, avulsions are all re-active forms of treatment, A preventive pro-active method to maintaining sanitary, dry and pH appropiate nails is introduced by the inventor here.

### BRIEF SUMMARY

The present invention relates to a nail sanitizing solution. More specifically, the present invention relates to a preventive method for sanitizing artificial fingernails and toe nails.

### DESCRIPTION OF THE INVENTION

Generally, the present invention provides an antiseptic composition/ sanitizing solution/cosmeceutical. More specifically, the antiseptic solution includes a drying compound and a sanitizer. The solution can be in a spray or gel soak form.

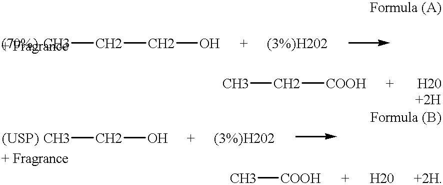

What is claimed is:

1. An artificial nail sanitizing solution for the reduction of nail contamination and subsequent microbial transfer.

2. The artificial nail sanitizing solution has the preferred composition of 2-propanol or isopropyl alcohol and 3% hydrogen peroxide. However, while peroxide is the preferred antiseptic/sanitizer others can be utilized. Such agents must be able to possess fungistatic or bacteriostatic properties when added to the composition.

3. The artificial nail sanitizing solution uses 2-propanol as the preferred drying agent, other drying agents can be used without departing from the purpose of the present invention. Examples of such drying agents are commonly known by those of skill in the art.

4. The artificial nail sanitizer wherein said solution has the ability to resist development of specific growth of skin flora organisms. Specifically molds, yeast, fungus and gram positive organisms will retard growth in an acidic environment, pH 3.0.

5. The artificial nail sanitizing solution when soaked; sprayed daily to periungual and subungual areas about the nail plate, is capable of establishing a barrier to the transmission of bacteria at the point of hand to hand contact.

6. The composition can include non-pharmaceutical additives, fragrance, aloe vera preparations, etc. The only requirement is that it must be compatible with both the antiseptic/sanitizer and drying agent and any products formed by the antiseptic/sanitizer and drying agent composition without affecting the properties of the composition. Such compositions are known by those of skill in the art.

7. The invention has been described in an illustrative manner and it is to be understood that the terminology which has been used is intended to be in the nature of words of description rather than of limitation.
Obviously, many modifications and variations of the present invention are possible in light of the above teachings. It is therefore, to be understood that within the scope of the described invention, the invention may be practiced otherwise than as specifically described.
